(19) United States
(12) Patent Application Publication
(10) Pub. No.: US 2024/0158420 A1
(43) Pub. Date: May 16, 2024

(54) [18F]A1F LABELED PSMA TARGETING MOLECULAR PROBE AND PREPARATION METHOD THEREFOR

(71) Applicants: FIVE ELEVEN PHARMA, INC, Philadelphia, PA (US); BIOMOLECULAR PROBES BIOTECHNOLOGY (BEIJING) CO., LTD, Beijing (CN)

(72) Inventors: Hank F KUNG, Philadelphia, PA (US); Zhihao ZHA, Beijing (CN); Karl PLOESSL, Philadelphia, PA (US); Seok Rye CHOI, Philadelphia, PA (US); David ALEXOFF, Philadelphia, PA (US)

(21) Appl. No.: 18/547,609

(22) PCT Filed: Mar. 17, 2021
(86) PCT No.: PCT/CN2021/078215

§ 371 Date: Aug. 23, 2023

(51) Int. Cl. C07F 5/06; G01N 33/574; G01N 33/68

(52) U.S. Cl. CPC C07F 5/069; G01N 33/57434; G01N 33/68

(57) ABSTRACT

A radiopharmaceuticals in clinical popularization is facilitated. By means of the biological characteristic of specifically targeting PSMA of the [¹⁸F]AlF-P16-093, the labeled product has important potential clinical values in the aspects of early diagnosis, preoperative staging, treatment guidance, recurrence and metastasis focus detection of prostate cancer.

### TECHNICAL FIELD OF THE INVENTION

The present invention relates to a novel ¹⁸F labeled positron emission tomography (PET) molecular probe and a preparation method therefor, and particularly, to an [¹⁸F]AlF labeled prostate specific membrane antigen (PSMA) targeting molecular probe and a preparation method therefor, and belongs to the technical field of radiolabeled compounds.

### BACKGROUND

Positron emission tomography (PET) technology is a tip technology in the field of contemporary nuclear medicine, and also one of the state-of-the-art technical means for human beings to study life phenomena in the 21st century. It is a non-invasive, dynamic, and quantitative imaging technique that utilizes positron emitting drugs as molecular probes to evaluate physiological and biochemical changes in vivo, and has been widely used in the differential diagnosis and efficacy monitoring of diseases such as tumors, cardiovascular and cerebrovascular diseases, and neurological diseases. Wherein positron drugs are the “soul” of PET imaging, and their development and clinical translation are the key to the development of PET diagnostic techniques. Positron drugs refer to drugs labeled with positron emitting radioactive nuclides for clinical PET imaging.

Positron nuclides commonly used for PET imaging are ¹¹C (half-life T_{1/2}=20.3 min), ¹⁸F (half-life T_{1/2}=109.8 min), ⁶⁸Ga (half-life T_{1/2}=68.1 min), etc. ¹⁸F is currently the most widely used positron nuclide in clinical application, and has good nuclear chemistry properties. ¹⁸F labeled compound has been the focus of positron molecular probe development.

Prostate cancer is a urinary malignancy severely threatening the health of elderly men. With the development of population aging and popularization of prostate specific antigen (PSA) screening, incidence and mortality of prostate cancer in China grows year-by-year, and metastatic castration-resistant prostate cancer resulting from progressive prostate cancer is a leading cause of death. Early and accurate diagnosis of prostate cancer patients may allow for timely therapeutic interventions and personalized therapeutic regimens and thus reduce the mortality of prostate cancer.

Prostate-specific membrane antigen (PSMA) is a highly specific epithelial cell membrane antigen of the prostate, and it is an important diagnostic and therapeutic target for prostate cancer. PET imaging technology has the characteristics of precision and non-invasive. In recent years, various ⁶⁸Ga-labeled PSMA-targeted PET imaging drugs have been reported in literature, represented by the unpatented ⁶⁸Ga-PSMA-11. Hank F. Kung Laboratories developed a new generation of ⁶⁸Ga-labeled radiopharmaceutical P16-093 based on existing PSMA targeting molecular probes, FIG. 1-1 illustrates the structure of ⁶⁸Ga-PSMA-11; FIG. 1-2 illustrates the structure of ⁶⁸Ga-P16-093.

⁶⁸Ga-P16-093 is coordinated with ⁶⁸Ga³⁺ (coordination constant˜38.5) by using HBED-CC as a bifunctional linker, which labeling method is simple, high stability in vivo, has significant radioactive enrichment in tumor metastatic sites of patients and promising properties for imaging. Phase I/II clinical study data showed that the patient did not experience significant toxic side effects after injection of ⁶⁸Ga-P16-093. Compared with ⁶⁸Ga-PSMA-11, ⁶⁸Ga-P16-093 showed a significant decrease in radiation uptake in urinary organs such as the bladder, and PET/CT imaging of the lesion site were more clear. As a novel PSMA targeting molecular probe, ⁶⁸Ga-P16-093 can be used in the diagnosis and research of prostate cancer and kidney cancer. It has entered phase II clinical trial in United States (Clinical Trials. gov Identifier: NCT034444444).

Compared with ⁶⁸Ga, ¹⁸F is currently the most widely used positron nuclide in clinical application and has good nuclear chemistry properties. ¹⁸F labeled compounds have always been the focus in the development of positron molecular probes.

To date, no researcher has conducted ¹⁸F labeling studies of P16-093. The inventor attempted to label P16-093 with ¹⁸F-AlF, and found that the labeling could be realized by adjusting the reaction conditions. The obtained ¹⁸F compound has superior biological properties and has the potential to be a new generation of PSMA positron molecular probes.

### SUMMARY OF THE INVENTION

An object of the present invention is to overcome the deficiencies of the prior art and provide a [¹⁸F]AlF labeled PSMA targeting molecular probe.

The aforementioned object of the present invention is realized through the following technical solutions:

A [¹⁸F]AlF labeled PSMA targeting molecular probe, comprises a PSMA-targeting group and the bifunctional linker HBED-CC, wherein the probe has a formula as below:

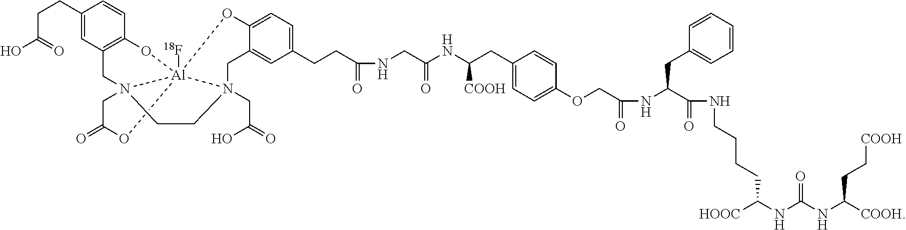

Another object of the present invention is to provide a preparation method for the aforementioned [¹⁸F]AlF labeled PSMA targeting molecular probe.

The aforementioned object of the present invention is realized by the following technical solutions:

A preparation method for the aforementioned [¹⁸F]AlF labeled PSMA targeting molecular probe, comprising the steps as below:
(1) P16-093 (PSMA-093) with the following structural formula was dissolved in sodium acetate buffer, and sodium hydroxide solution was added, adjusted the pH value to 4-6, and obtained a P16-093 sodium acetate solution;

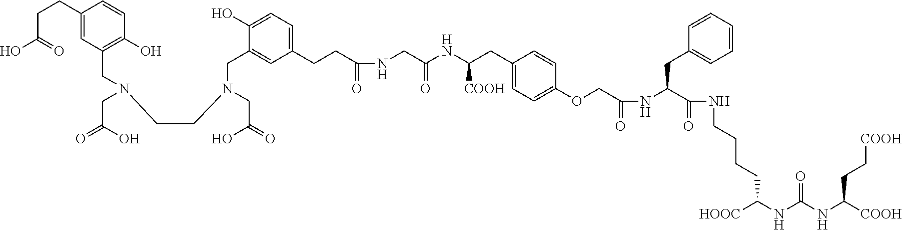 [SMILES: O=C(O)CCc1ccc(O)c(CN(CCN(CC(=O)O)Cc2cc(CCC(=O)NCC(=O)N[C@@H](Cc3ccc(OCC(=O)N[C@@H](Cc4ccccc4)C(=O)NCCCC[C@H](NC(=O)N[C@@H](CCC(=O)O)C(=O)O)C(=O)O)cc3)C(=O)O)ccc2O)CC(=O)O)c1]

(2) ¹⁸F⁻ was prepared on a cyclotron by nuclear reaction ¹⁸ O(p,n)¹⁸F, which was then enriched on WATERS anion exchange column (sep-pak QMA), rinsed with deionized water to remove the metal impurity ions adsorbed on the sep-pak QMA column, and eluted with saline to obtain a saline solution;
(3) A sodium acetate buffer of AlCl₃ was added in a reaction vessel, and the saline solution prepared in step (2) was added and mixed evenly, and then the P16-093 sodium acetate solution prepared in step (1) and ethanol were added, the mixture was shaken evenly and reacted at 50-80° C. for 10-30 min, then cooling to ambient temperature to obtain a [¹⁸F]AlF-P16-093 labeled reaction solution;
(4) The [¹⁸F]AlF-P16-093 labeled reaction solution prepared in step (3) was purified to obtain the [¹⁸F]AlF labeled PSMA targeting molecular probe.

Preferably, step (1) was as follows: 6 mg of P16-093 was dissolved in 2 mL of 0.05N sodium acetate buffer, and sodium hydroxide solution was added , adjusted the pH value to 5, and obtained a P16-093 sodium acetate solution at concentration of 3 mg/mL.

Preferably, step (2) was as follows: a sep-pak QMA column was pre-treated with 10 mL of 0.5M NaOAc solution and deionized water, and ¹⁸F⁻ was prepared on a cyclotron by nuclear reaction ¹⁸O(p,n)¹⁸F and then enriched on the sep-pak QMA column, rinsed with deionized water to remove the metal impurity ions adsorbed on the sep-pak QMA column, and eluted with 0.2˜1 mL of saline to obtain 1˜2 GBq of a saline solution.

Preferably, step (3) was as follows: 4 μL of sodium acetate buffer of AICl₃ was added in a reaction vessel, and 100 μL of the saline solution prepared in step (2) was added and mixed evenly, and then the P16-093 sodium acetate solution prepared in step (1) and 124 μL of ethanol were added. The mixture was shaken evenly and reacted at 50-80° C. for 10-30 min, then cooling to ambient temperature, the labeling yield was measured by high performance liquid chromatography (HPLC) to obtain a [¹⁸F]AlF-P16-093 labeled reaction solution.

Preferably, step (4) was as follows: the [¹⁸F]AlF-P16-093 labeled reaction solution prepared in step (3) was purified by solid phase extraction small column, the resulting product was diluted with saline to an ethanol content of less than 10%, its retention time and radiochemical purity were measured by HPLC, and its appearance was observed as a colorless clear transparent liquid to obtain a [¹⁸F]AlF labeled PSMA targeting molecular probe.

Preferably, in step (3) and step (4), the first mobile phase was 0.1% aqueous solution of trifluoroacetic acid, the second mobile phase was acetonitrile, the gradient elution conditions were as follows: 0 min, 100% of the first mobile phase; 0˜10 min, 100% ˜0% of first mobile phase; the flow rate of the mobile phase was 1 ml/min.

A further object of the present invention is to provide the use of the aforementioned [¹⁸F]AlF labeled PSMA targeting molecular probe.

The aforementioned object of the present invention is realized by the following technical solutions:

Use of [¹⁸F]AlF labeled PSMA targeting molecular probe in the diagnosis and detection of prostate cancer.

Preferably, the diagnosis and detection include early diagnosis, preoperative staging, treatment guidance, recurrence, and metastatic lesion detection.

### Benefits:

1. Based on the molecular probe P16-093 which has entered Phase II clinical studies in the United States, the PSMA targeting molecular probe AlF-P16-093 is designed to be radiolabeled without affecting its biological activity, ¹⁸F is used as a radioactive nuclide, and the PSMA targeting molecular probe is labeled with the method of [¹⁸F]AlF-HBED. The obtained [^{˜}F]AlF labeled PSMA targeting molecular probe has excellent pharmacokinetic properties, high stability in vivo, has significant radioactive enrichment in tumor metastatic sites of patients, low bladder uptake, and has superior properties for imaging, which is more conducive to the detection of tumor lesions in pelvic location; moreover, the subject does not need to promote urination of bladder for imaging; in addition, the highest tumor uptake could be reached at 6 min post-injection, and the imaging time could be advanced to around 10 min post-injection (other ⁶⁸Ga-labeled or ¹⁸F-labeled PSMA imaging agents need to be imaged 1˜2 h post-injection), with clinically potential application value for local lesion detection and rapid imaging.
2. The aforementioned [¹⁸F]AlF labeled PSMA targeting molecular probe, by means of the biological characteristic of specifically targeting PSMA, has important clinical potential values in early diagnosis, preoperative staging, treatment guidance, recurrence, and metastasis focus detection of prostate cancer.
3. The aforementioned [¹⁸F]AlF labeled PSMA targeting molecular probe is radiolabeled by means of [¹⁸F]AlF-HBED. The labeling method is simple, automatic synthesis is easy to realize, the labeling yield is high, HPLC purification is not required, which is important for radionuclides with shorter half-lives and is more conducive to commercial application of the radiolabeled compound in clinical promotion.

The subject matter of the present invention is further illustrated and described in connection with the example and the corresponding text and figures. There is no intent to limit the protection scope of the present invention.

### BRIEF DESCRIPTION OF THE DRAWINGS

FIG. 1-1 illustrates the structure of ⁶⁸Ga-PSMA-11.

FIG. 1-2 illustrates the structure of ⁶⁸Ga-P16-093.

FIG. 2-1 illustrates the radioactivity peak chromatogram of [¹⁸F]AlF-P16-093, with time in abscissa and radioactivity peak absorption intensity in ordinate.

FIG. 2-2 illustrates the UV absorption spectrum of AlF-P16-093 standard, with time in abscissa and UV absorption intensity in ordinate.

FIG. 3-1 illustrates the results of autoradiography study of [¹⁸F]AlF-P16-093 in Example 1 of the present invention in PC3-PIP tumors, with the top half illustrates autoradiography without inhibitor and the bottom half illustrates autoradiography with inhibitor MIP-1095.

FIG. 3-2 illustrates the results of autoradiography study of [¹⁸F]AlF-P16-093 in Example 1 of the present invention in PC3 tumors, with the top half illustrates autoradiography without inhibitor and the bottom half illustrates autoradiography with inhibitor MIP-1095.

FIG. 3-3 illustrates the results of autoradiography study of [¹⁸F]AlF-P16-093 in Example 1 of the present invention in kidney tissues, the top half illustrates autoradiography without inhibitor and the bottom half illustrates autoradiography with inhibitor MIP-1095.

FIG. 4 illustrates the results of an uptake study of [¹⁸F]AlF-P16-093 in Example 1 of the present invention in PC3-PIP cells and PC-3 cells.

### DETAILED DESCRIPTION

Unless otherwise specified, the raw material and reagents mentioned in the examples of the present disclosure are the conventional ones that are commercially available, the test methods used are conventional, and the equipment and devices used are all conventional ones in this field.

A preparation method of the [¹⁸F]AlF labeled PSMA targeting molecular probe, comprising the following steps:
(1) 100 mg of aluminum trifluoride and 5 mg of P16-093 (the structure formula was as follows) were dissolved in 2 mL of 0.05N sodium acetate solution buffer and 200 μL of ethanol, reacted at 80° C. for 30 min, cooled to ambient temperature, and purified by prep-HPLC to obtain the standard compound of AlF-P16-093, the purity was measured by high performance liquid chromatography (HPLC);

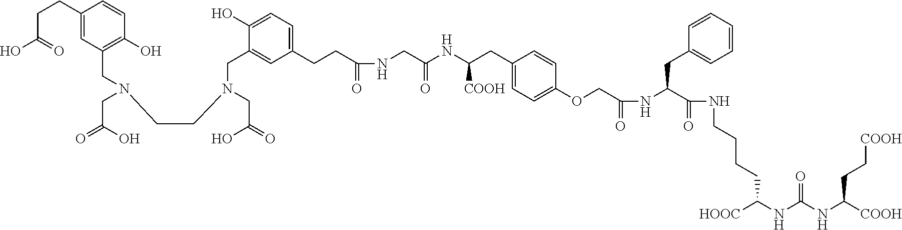 [SMILES: O=C(O)CCc1ccc(O)c(CN(CCN(CC(=O)O)Cc2cc(CCC(=O)NCC(=O)N[C@@H](Cc3ccc(OCC(=O)N[C@@H](Cc4ccccc4)C(=O)NCCCC[C@H](NC(=O)N[C@@H](CCC(=O)O)C(=O)O)C(=O)O)cc3)C(=O)O)ccc2O)CC(=O)O)c1]

### The Structure of P16-093

(2) 6 mg of P16-093 (PSMA-093, supplied by Five Eleven Pharma, Inc., USA) was dissolved in 2 mL of 0.05N sodium acetate buffer, and sodium hydroxide solution was added, adjusted the pH value to 5 and obtained a P16-093 solution at concentration of 3 mg/mL;
(3) Sep-pak QMA column was pre-treated with 10 mL of 0.5M NaOAc solution and deionized water, and ¹⁸F⁻ was prepared on a cyclotron by nuclear reaction ¹⁸O(p,n)¹⁸F and then enriched on the sep-pak QMA column, rinsed with deionized water to remove the metal impurity ions adsorbed on the column , and eluted with 0.2˜1 mL of saline to obtain 1˜2 GBq of a saline solution;
(4) 4 μL of AlCl₃ (4 nmol) sodium acetate buffer was added to a reaction vessel, then 100 μL of saline solution prepared in step (3) was added and mixed evenly, and then P16-093 sodium acetate solution prepared in step (2) and 124 μL of ethanol were added, the mixture was shaken evenly and reacted at 50 to 80° C. for 10 to 30 min, after cooling to ambient temperature, the labeling yield was measured by HPLC to obtain a [¹⁸F]AlF-P16-093 labeling solution;
(5) 6 mL of deionized water was added to the [¹⁸F]AlF-P16-093 labeling reaction prepared in step (4) and mixed evenly, then purified by solid phase extraction small column, the resulting eluant was diluted with saline to the ethanol content of less than 10%, its appearance was observed as a colorless clear transparent liquid, and its retention time and radiochemical purity were measures by HPLC, and comparative analysis was performed with AlF-P16-093 standard, the consistent results indicating that a [¹⁸F]AlF labeled PSMA targeting molecular probe was obtained, which has the following structure:

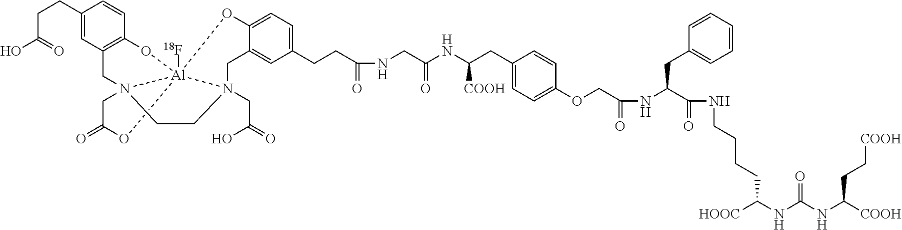

In high performance liquid chromatography (HPLC) aforementioned in step (1), step (4) and step (5), the first mobile phase was 0.1% aqueous solution of trifluoroacetic acid, the second mobile phase was acetonitrile, the gradient elution conditions were as follows: Omin, 100% of the first mobile phase; 0˜10 min, 100% ˜0% of first mobile phase; the flow rate of the mobile phase was 1 ml/min.

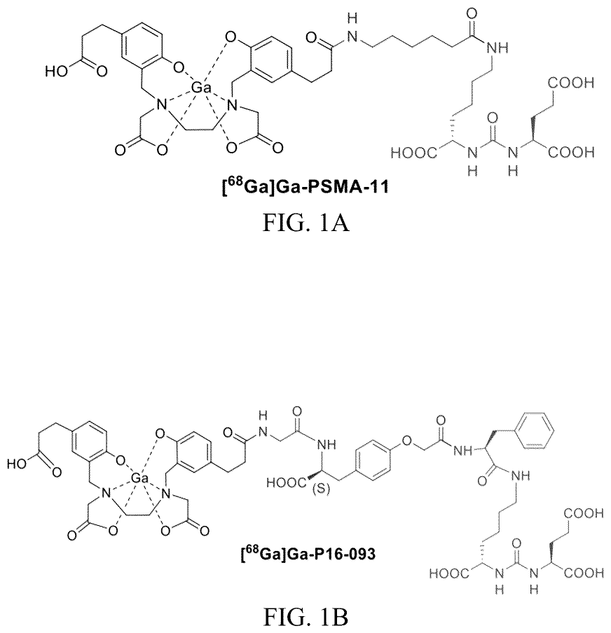

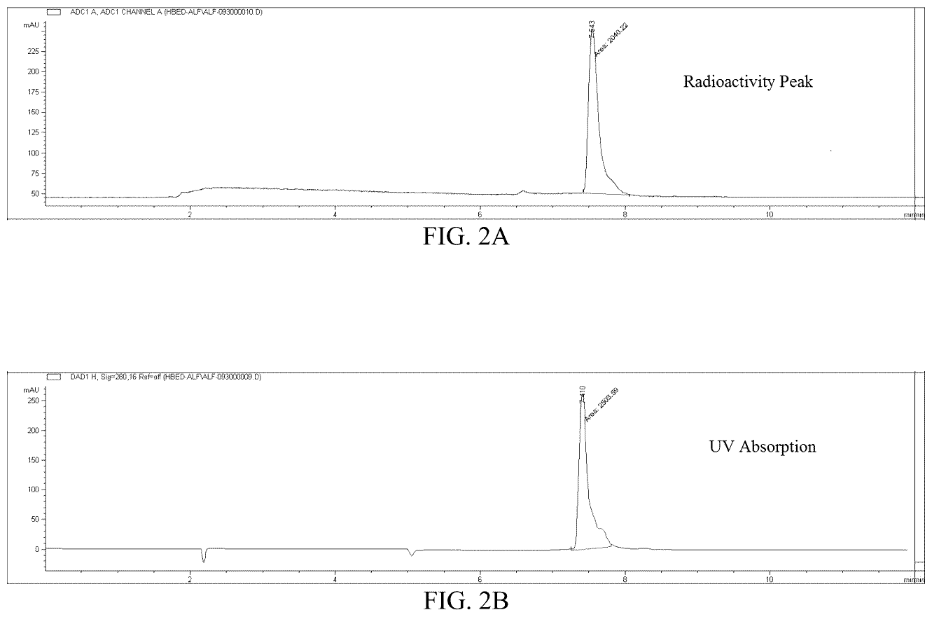

FIG. 2-1 illustrates the radioactivity peak chromatogram of [¹⁸F]AlF-P16-093, with time in abscissa and radioactivity peak absorption intensity in ordinate; FIG. 2-2 illustrates the UV absorption spectrum of AlF-P16-093 reference standard, with time in abscissa and UV absorption intensity in ordinate.

### Application Example 1: In Vitro Autoradiography Experiments

Tumor mouse models of PC3-PIP (overexpressing of PSMA) and PC3 (no expressing of PSMA) were established, the PC3-PIP tumor, PC3 tumor and kidney tissue of the tumor mouse were sectioned at −20° C. with a thickness of 20 μm, the obtained sections were placed on a glass slide and dried in air for in vitro autoradiography studies. 1 mL of [¹⁸F]AlF-P16-093 solution was overlaid on the tissue sections, incubated at room temperature for 60 minutes, and rinsed with PBS solution and deionized water for 3 minutes respectively, after drying, the tissue sections were covered with cling film and exposed overnight on a phosphorscreen, and then placed in a Cyclone Storage Phosphor System for imaging analysis.

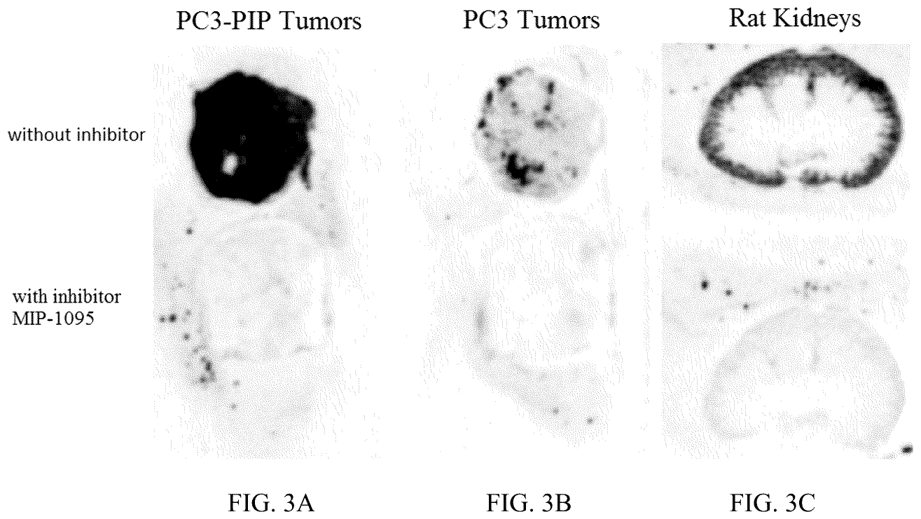

The results of in vitro autoradiography experiments are illustrated in FIG. 3-1 to FIG. 3-3, FIG. 3-1 illustrates the results of autoradiography study of [¹⁸F]AlF-P16-093 in Example 1 of the present invention in PC3-PIP tumors, with the top half illustrates autoradiography without inhibitor and the bottom half illustrates autoradiography with inhibitor MIP-1095; FIG. 3-2 illustrates the results of autoradiography study of [¹⁸F]AlF-P16-093 in Example 1 of the present invention in PC3 tumors, with the top half illustrates autoradiography without inhibitor and the bottom half illustrates autoradiography with inhibitor MIP-1095; FIG. 3-3 illustrates the results of autoradiography study of [¹⁸F]AlF-P16-093 in Example 1 of the present invention in kidney tissues, with the top half illustrates autoradiography without inhibitor and the bottom half illustrates autoradiography with inhibitor MIP-1095; [¹⁸F]AlF-P16-093 was highly concentrated on PC3-PIP tumor and kidney sections (expressing PSMA), and no apparent uptake on PC3 tumor sections (not expressing PSMA).

### Application Example 2: Cellular Uptake Experiments

PC3-PIP cells (expressing PSMA) and PC-3 cells (no expressing PSMA) were seeded separately in 12-well plates, RPMI 1640 culture fluid and fetal bovine serum FBS (v/v=9/1) were added, after incubation in a CO2 incubator (5% of CO₂, 37° C.) for 24 hours, there were approximately 5×10⁵ cells per well plate, the medium was moved, and washed twice with PBS, then PBS solution of [¹⁸F]AlF-P16-093 or RPMI 1640 culture fluid was added, after incubation in a CO2 incubator (5% of CO₂, 37° C.) for 0-120 minutes, the solution was removed, washed twice with 1 mL of cold PBS (without Ca2+ and Mg2+), the cells and cold PBS radioactive count were measured by a γ-Counter (radioactive counting instrument). Simultaneously, inhibition experiments were conducted, 10 μM MIP-1095 was added per well plate.

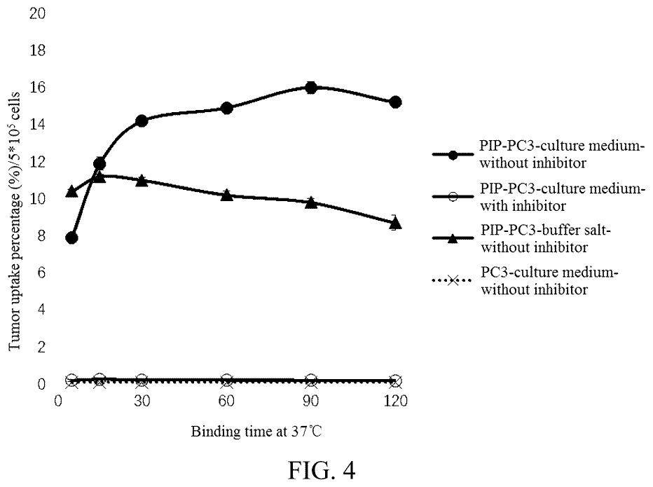

The results of the cellular uptake studies of [¹⁸F]AlF-P16-093 in Example 1 of the present invention in PC3-PIP cells and PC-3 cells are illustrated in FIG. 4: higher uptake of [¹⁸F]AlF-P16-093 in PC3-PIP cells, and the uptake is gradually increased over time; low uptake (<0.1%) in PC-3 cells, which indicates that [¹⁸F]AlF-P16-093 had specific uptake for PMSA.

### Application Example 3: In Vivo Biodistribution Study In Tumor Mice

CD-I nude mice were implanted with PC3-PIP cells in the anterior left extremity axilla and PC3 cells in the anterior right extremity axilla and grown until tumors were 5-8 mm in diameter for biodistribution studies, by tail vein injection of 0.15 mL of [¹⁸F]AlF-P16-093 saline (approximately 370 kBq), and tumor bearing nude mice were sacrificed anesthetically at different time points (30min, 60 min and 120 min), dissected, tissue of interest weighed and radioactivity counted;

The results of in vivo biodistribution studies in tumor mice are illustrated in Table 1: [¹⁸F]AlF-P16-093 in vivo biodistribution studies in tumor mice (% dose/g, avg±SD, n=4); it can be seen that [¹⁸F]AlF-P16-093 uptake was higher in kidneys and PC3-PIP tumors of PSMA-high expressing, rising stepwise with time and reaching a maximum at 60 min; the uptake was lower in PC3 tumors which PSMA did not express; PC3-PIP tumor to muscle ratios were 17, 43 and 63 at 30 min, 60 min and 120 min respectively; PC3-PIP tumor to blood ratios were 11, 29 and 61 respectively.

TABLE 1

| | 30 m | 60 m | 120 m | 60 m Blocking* |
|---|---|---|---|---|
| Blood | 1.30 ± 0.24 | 0.68 ± 0.09 | 0.29 ± 0.02 | 0.50 ± 0.24 |
| Cardiac | 1.53 ± 0.43 | 0.82 ± 0.14 | 0.49 ± 0.08 | 0.35 ± 0.11 |
| Muscle | 0.89 ± 0.22 | 0.44 ± 0.03 | 0.29 ± 0.05 | 0.34 ± 0.19 |
| Lung | 2.75 ± 0.53 | 1.80 ± 0.22 | 1.23 ± 0.13 | 0.63 ± 0.21 |
| Kidney | 78.84 ± 13.64 | 98.57 ± 12.59 | 102.25 ± 16.97 | 2.11 ± 1.04 |
| Spleen | 7.08 ± 3.88 | 4.54 ± 1.52 | 2.52 ± 0.41 | 0.30 ± 0.19 |
| Liver | .69 ± 0.38 | 1.12 ± 0.15 | 0.71 ± 0.13 | 0.64 ± 0.28 |
| Bone | 2.92 ± 0.42 | 2.83 ± 0.49 | 4.20 ± 0.54 | 11.40 ± 1.61 |
| PIP-PC3 Tumors | 14.80 ± 4.82 | 18.84 ± 5.14 | 17.66 ± 3.34 | 2.15 ± 0.58 |
| PC3 Tumors | 1.87 ± 0.36 | 1.49 ± 0.03 | 1.11 ± 0.05 | 0.67 ± 0.27 |
| PIP-PC3/Blood | 11.26 ± 2.43 | 28.92 ± 12.11 | 60.96 ± 12.59 | 4.92 ± 2.59 |
| PIPPC3/Muscle | 16.68 ± 4.03 | 42.80 ± 13.13 | 63.39 ± 15.32 | 7.49 ± 3.83 |

In the present invention, ¹⁸F is used as a radioactive labeled nuclide, and a PSMA targeting molecular probe is labeled by means of [¹⁸F]AlF-HBED. The labeled [¹⁸F]AlF-P16-093 has two advantages: firstly, the labeled method of [¹⁸F]AlF-P16-093 is simple, evaporation and water removal are not required, automatic synthesis is easy to realize, the labeling yield is high, HPLC purification is not required, and commercial application of radiopharmaceuticals in clinical promotion is facilitated; and secondly, by means of the biological characteristic of specifically targeting PSMA of the [¹⁸F]AlF -P16-093, the labeled product has important potential clinical values in early diagnosis, preoperative staging, treatment guidance, recurrence and metastasis focus detection of prostate cancer.

It is to be understood that the described examples herein are only some preferred examples of the present invention, there is no intent to limit the scope of implementation of the present invention. Equivalent changes and modifications made according to the patent scope and specification of the present invention should still fall within the scope of the present invention.

1. A [¹⁸F]AlF labeled PSMA targeting molecular probe, which has the following chemical structure:

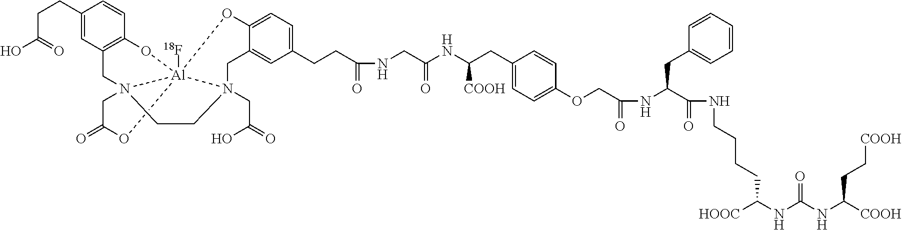

2. A preparation method of the [¹⁸F]AlF labeled PSMA targeting molecular probe, comprising the following steps:
(1) dissolving P16-093 with the following structure in sodium acetate buffer, adding sodium hydroxide solution, adjusted the pH value to 4-6 and obtained a P16-093 sodium acetate solution; 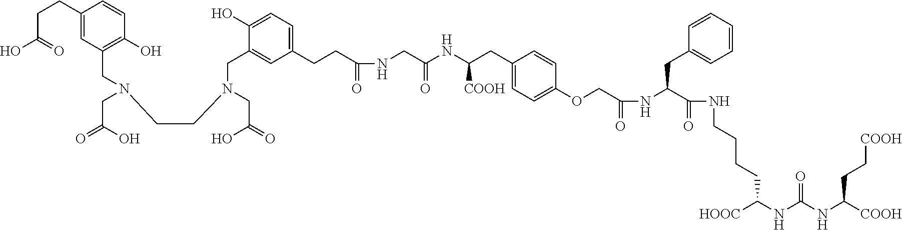 [SMILES: O=C(O)CCc1ccc(O)c(CN(CCN(CC(=O)O)Cc2cc(CCC(=O)NCC(=O)N[C@@H](Cc3ccc(OCC(=O)N[C@@H](Cc4ccccc4)C(=O)NCCCC[C@H](NC(=O)N[C@@H](CCC(=O)O)C(=O)O)C(=O)O)cc3)C(=O)O)ccc2O)CC(=O)O)c1]
(2) ¹⁸F⁻ is prepared on a cyclotron by nuclear reaction ¹⁸O(p,n)¹⁸F, which is then enriched on WATERS anion exchange resin column, rinsing with deionized water to remove the metal impurity ions adsorbed on the column, and eluting with saline to obtain a saline solution;
(3) adding the sodium acetate buffer of AlCl₃ in a reaction vessel, and adding the saline solution prepared in step(2) and mixing evenly, and then adding the P16-093 sodium acetate solution prepared in step(1) and ethanol, shaking the mixture evenly and reacting at 50-80° C. for 10-30min, then cooling the mixture to ambient temperature to obtain the [¹⁸F]AlF-P16-093 labeled reaction solution;
(4) purifying the [¹⁸F]AlF-P16-093 labeled reaction solution prepared in step(3) to obtain the [¹⁸F]AlF labeled PSMA targeting molecular probe.

3. The preparation method of the [¹⁸F]AlF labeled PSMA targeting molecular probe according to claim 2, characterized in that: step (1) is as follows: dissolving 6 mg of P16-093 in 2 mL of 0.05N sodium acetate buffer, then adding sodium hydroxide solution, adjusted the pH value to 5 and obtained a P16-093 solution at concentration of 3 mg/mL.

4. The preparation method of the [¹⁸F]AlF labeled PSMA targeting molecular probe according to claim 3, characterized in that: Step (2) is as follows: WATERS anion exchange resin column is pretreated with 10 mL of 0.5M NaOAc solution and deionized water, and ¹⁸F⁻ is prepared on a cyclotron by nuclear reaction ¹⁸O(p,n) ¹⁸ F and then enriched on WATERS anion exchange resin column, rinsing with deionized water to remove the metal impurity ions adsorbed on the column, and eluting with 0.2-1 mL saline to obtain 1-2 GBq of a saline solution.

5. The preparation method of the [¹⁸F]AlF labeled PSMA targeting molecular probe according to claim 4, characterized in that: Step (3) is as follows: adding 4 μL of a sodium acetate buffer of A1C13 to a reaction vessel, then adding 100 μL of the saline solution prepared in step (2) and mixing evenly, and then adding the P16-093 sodium acetate solution prepared in Step (1) and 124 μL of ethanol, shaking the mixture evenly and reacting at 50-80° C. for 10-30 min, cooling the mixture to ambient temperature, and measuring the labeling yield by HPLC to obtain the [¹⁸F]AlF-P16-093 labeled reaction solution.

6. The preparation method of the [¹⁸F]AlF labeled PSMA targeting molecular probe according to claim 5, characterized in that: Step (4) is as follows: purifying the [¹⁸F]AlF-P16-093 labeled reaction solution prepared in step (3) by solid phase extraction small column, diluting the resulting product with saline to the ethanol content of less than 10%, measuring its retention time and radiochemical purity by HPLC, and observing its appearance as a colorless clear transparent liquid to obtain the [¹⁸F]AlF labeled PSMA targeting molecular probe.

7. The preparation method of the [¹⁸F]AlF labeled PSMA targeting molecular probe according to claim 8, characterized in that: in the HPLC of step (3) and step (4), the first mobile phase is 0.1% aqueous solution of trifluoroacetic acid, the second mobile phase is acetonitrile, the gradient elution conditions are as follows: 0 min, 100% of the first mobile phase; 0˜10 min, 100%˜0% of first mobile phase; the flow rate of the mobile phase was 1 ml/min.

8. A method for diagnosis and detection of prostate cancer, comprising administering the [¹⁸F]AlF labeled PSMA targeting molecular probe according to claim 1 to a subject in need thereof.

9. The method according to claim 8, wherein the diagnosis and detection of prostate cancer comprise early diagnosis, preoperative staging, treatment guidance, recurrence or metastatic lesion detection.
